(19) United States
(12) Patent Application Publication
(10) Pub. No.: US 2018/0339090 A1
(43) Pub. Date: Nov. 29, 2018

(54) THERAPEUTIC SUCTION CUP

(71) Applicant: Michael Joseph Santana, Montvale, NJ (US)

(72) Inventor: Michael Joseph Santana, Montvale, NJ (US)

(21) Appl. No.: 15/959,460

(22) Filed: Apr. 23, 2018

Related U.S. Application Data
(60) Provisional application No. 62/511,310, filed on May 25, 2017.

(51) Int. Cl. A61M 1/08

(52) U.S. Cl. CPC A61M 1/08

(57) ABSTRACT

A therapeutic suction cup. The therapeutic suction cup includes a body having a dome shape and a sealing rim. The therapeutic suction cup further includes a handle attached to an outer surface of the body. The handle allows a user to manipulate the suction cup when the suction cup is vacuum attached to skin of a patient.

### CROSS-REFERENCE TO RELATED APPLICATION

This application claims the benefit of priority of U.S. provisional application No. 62/511,310, filed May 25, 2017, the contents of which are herein incorporated by reference.

### BACKGROUND OF THE INVENTION

The present invention relates to therapeutic suction cups and, more particularly, to a therapeutic suction cup that can be manipulated once the cup is applied to the skin.

Cupping therapy is a form of alternative medicine in which a local suction is created on the skin. Through suction, the skin is drawn into the cup by creating a vacuum in the cup placed on the skin over the targeted area. The vacuum can be created either by the heating and subsequent cooling of the air in the cup, or via a mechanical pump. The cup is usually left in place for somewhere between five and fifteen minutes and helps treat pain, deep scar tissues in the muscles and connective tissue, muscle knots, and swelling. Current therapeutic cups do not allow for easy manipulation once the cup has been vacuum attached to the skin.

As can be seen, there is a need for a therapeutic suction cup that can be manipulated once the cup is applied to the skin.

### SUMMARY OF THE INVENTION

In one aspect of the present invention, a therapeutic suction cup comprises: a body comprising a dome shape; a sealing rim; and a handle attached to an outer surface of the body.

In another aspect of the present invention, a method of providing suction cup therapy comprises steps of: providing at least one suction cup comprising: a body comprising a dome shape; a sealing rim; and a handle attached to an outer surface of the body; placing the sealing rim against a skin of a patient; creating a vacuum within the body, thereby securing the at least one suction cup to the skin; and manipulating the at least one suction cup via the handle.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is an exploded view of an embodiment of the present invention;

FIG. 3 is a front view of an embodiment of the present invention;

FIG. 4 is a section view of the present invention, taken along line 4-4 in FIG. 3, illustrating the flexibility of the handle; and

FIG. 5 is a perspective view of an embodiment of the present invention shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a system to allow for the application of tension against the skin to which a vacuum is applied under a therapeutic vacuum cup. This tension has the effect of lifting the skin under the vacuum to be lifted up, thereby spreading the force of the vacuum exerted under the cup to the skin adjacent to the cup. The tension then has the further effect of slightly countering the natural effect of gravity.

The tension is introduced into the therapeutic vacuum cup system by means of the attachment of a handle to a therapeutic vacuum cup. The handle has the shape of an upside-down “U”, with the point of the inverted “U” lying along the line of the center of the vacuum force applied against the skin under the therapeutic vacuum cup. Thus, when a cord is attached to the top-most point of the inverted-U, the cord may then be attached to a fixed object, typically a therapy cage, and tension is then applied to the line attached to the inverted-U shaped handle.

The handle in the shape of an inverted-U may be created by means of fashioning a plastic tube, flattened at both ends by the application of heat and then pressure; and which is then attached to the therapeutic vacuum cup.

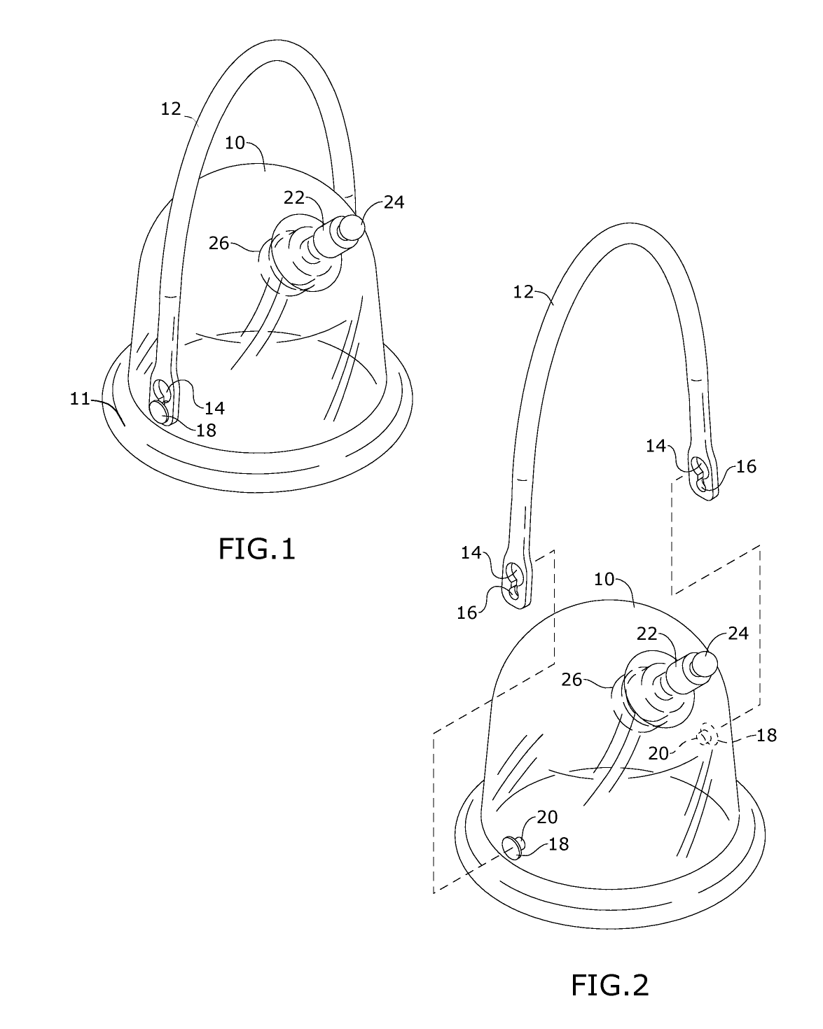

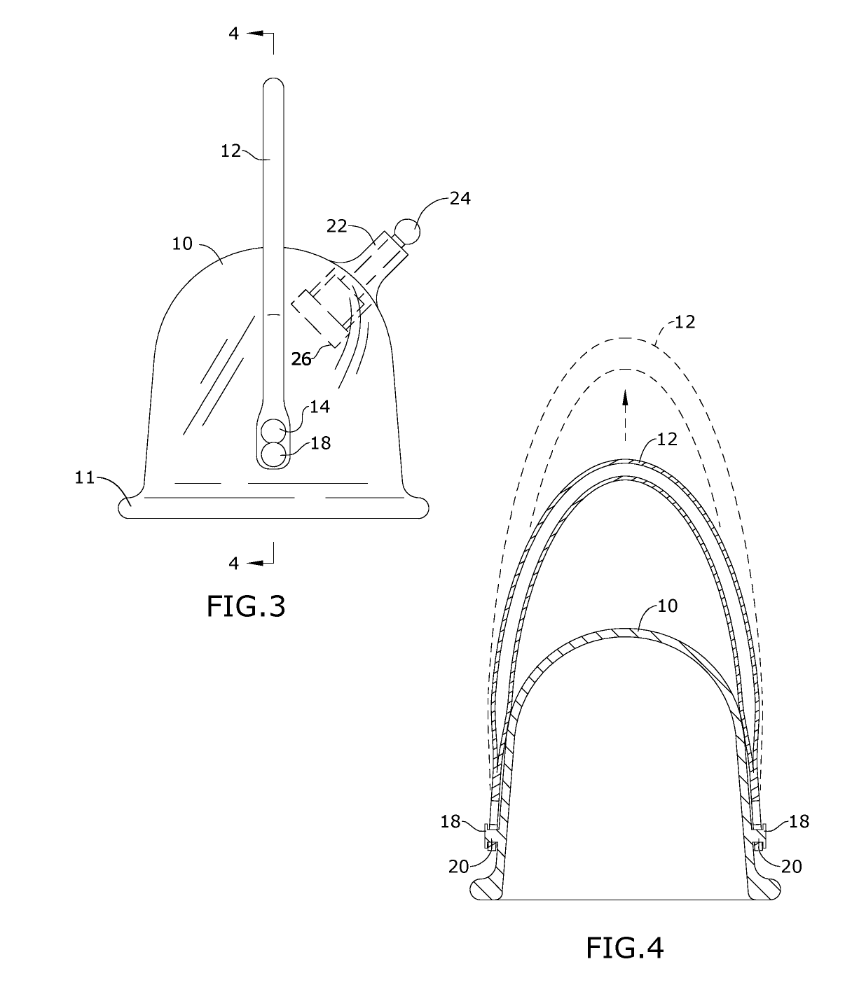

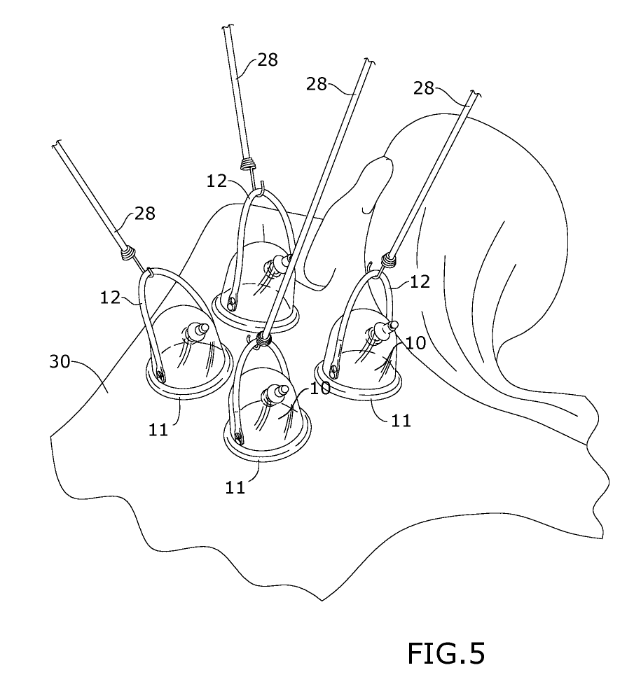

Referring to FIGS. 1 through 5, the present invention includes a therapeutic suction cup. The therapeutic suction cup includes a body 10 having a dome shape and a sealing rim 11. The sealing rim 11 may extend radially and circumferentially from a bottom edge of the body 10. The therapeutic suction cup further includes a handle 12 attached to an outer surface of the body 10. The handle 12 allows a user to manipulate the suction cup when the suction cup is vacuum attached to skin of a patient 30.

The body 10 and the sealing rim 11 may be made out of glass or a strong plastic. A vacuum is created within the body 10 when the sealing rim 11 is pressed against the skin of a patient 30. The vacuum may be created by applying heat to the body 10. Alternatively, a valve 22 may be used to create the vacuum within the body 10 by selectively removing air within the body 10. The valve 22 may be attached to the body 10. The valve 22 may include a piston 26 and a rod 24. The rod 24 may slide within the piston 26 and may be used to pump air out of the body 10.

The handle 12 of the present invention may have a U-shape having a first end attached to a first side of the body 10 and a second end attached to a second side of the body 10 opposite the first side. The handle 12 may be made of a material comprising a rubber elasticity, such as a flexible plastic, silicone, rubber and the like. In certain embodiments, the first end and the second end of the handle 12 are releasably attached to the body 10. In such embodiments, couplers 18, 20 extend from the first side and the second side of the body 10. The first end and the second end of the handle 12 releasably attaches to the couplers 18. 20.

In certain embodiments, the couplers 18, 20 each include a narrow elongated shaft 20 and a wide flat cap 18. Each of the ends of the handle 12 may include a key hole 14, 16. The key hole 14, 16 includes a wide opening 14 contiguous with a narrow opening 16. The narrow opening 16 may be beneath the wide opening 14. The wide opening 14 is sized to fit over the cap 18. Once the cap 18 is through the wide opening 14, the handle 12 is pulled upwards so that the shaft 20 is disposed within the narrow opening 16, thereby releasably attaching the handle 12 to the body 10.

A method of providing suction cup therapy may include the following steps: providing at least one suction cup described above; placing the sealing rim 11 against a skin of a patient 30; creating a vacuum within the body 10, thereby securing the at least one suction cup to the skin; and manipulating the at least one suction cup via the handle 12. The step of manipulating the at least one suction cup via the handle 12 may include the step of pulling the handle 12 away from the patient 30. The present invention may further include releasably attaching a cord 28 to the handle 12 via a hook. The cord 28 may be suspended above the patient 30. A patient 30 may be treated with a plurality of suction cups. A cord 28 may be releasably attached to each of the plurality of suctions cups. The cords 28 may be attached to a fixed object, such as a therapy cage, and tension may be applied to the cord 28 which pulls the handle 12.

It should be understood, of course, that the foregoing relates to exemplary embodiments of the invention and that modifications may be made without departing from the spirit and scope of the invention as set forth in the following claims.

What is claimed is:

1. A therapeutic suction cup comprising:
a body comprising a dome shape;
a sealing rim; and
a handle attached to an outer surface of the body.

2. The therapeutic suction cup of claim 1, wherein the handle comprises a U-shape having a first end attached to a first side of the body and a second end attached to a second side of the body, opposite the first side.

3. The therapeutic suction cup of claim 2, wherein the handle is made of a material comprising a rubber elasticity.

4. The therapeutic suction cup of claim 2, wherein the first end and the second end of the handle are releasably attached to the body.

5. The therapeutic suction cup of claim 4, wherein the body comprises couplers extending from the first side and the second side of the body, wherein the first end and the second end releasably attach to the couplers.

6. The therapeutic suction cup of claim 5, wherein the couplers each comprise a post and a cap, wherein each of the first end and the second end of the handle comprise a key hole sized to mate with the post and the cap.

7. The therapeutic suction cup of claim 1, further comprising a valve selectively allowing air in and out of the body.

8. The therapeutic suction cup of claim 7, wherein the valve comprises a valve piston and a valve rod.

9. A method of providing suction cup therapy comprising steps of:
providing at least one suction cup comprising:
a body comprising a dome shape;
a sealing rim; and
a handle attached to an outer surface of the body;
placing the sealing rim against a skin of a patient;
creating a vacuum within the body, thereby securing the at least one suction cup to the skin; and
manipulating the at least one suction cup via the handle.

10. The method of claim 9, wherein the step of manipulating the at least one suction cup via the handle comprises the step of pulling the handle away from the patient.

11. The method of claim 9, further comprising the step of releasably attaching a cord to the handle, wherein the cord is suspended above the patient.

12. The method of claim 9, wherein the handle comprises a U-shape having a first end attached to a first side of the body and a second end attached to a second side of the body, opposite the first side.

13. The method of claim 9, wherein the handle is made of a material comprising a rubber elasticity.

14. The method of claim 9, wherein the first end and the second end of the handle are releasably attached to the body.
